(19) United States
(12) Patent Application Publication
(10) Pub. No.: US 2024/0172755 A1
(43) Pub. Date: May 30, 2024

(54) USE OF PORPHYRIN NANOSTRUCTURES AS ANTIMICROBIAL AGENTS

(71) Applicant: LUNANO INC., McLean, VA (US)

(72) Inventors: Thomas WEBSTER, McLean, VA (US); Bradley DUCKWORTH, McLean, VA (US); Nasuh ONAL, McLean, VA (US)

(21) Appl. No.: 18/282,041

(22) PCT Filed: Mar. 22, 2022
(86) PCT No.: PCT/US2022/021238

§ 371 Date: Sep. 14, 2023

Related U.S. Application Data
(60) Provisional application No. 63/164,009, filed on Mar. 22, 2021.

(51) Int. Cl. A01N 55/02; A01N 33/12; A01P 1/00

(52) U.S. Cl. CPC A01N 55/02; A01N 33/12; A01P 1/00

(57) ABSTRACT

Methods of reducing or inhibiting growth of microbes (viruses, fungi and/or bacteria, including multi-drug resistant bacteria) can be carried out by applying a formulation including porphyrin nanoparticles to a surface to impart a residue of the porphyrin nanoparticles on the surface. The applied porphyrin nanoparticles residue can reduce or inhibit growth of infectious microbes on the surface for an extended period of time such as for at least 24 hours, 2 days, one week, etc. The applied porphyrin nanoparticles have high antimicrobial activity under visible light and without visible light.

### CROSS-REFERENCE TO RELATED APPLICATION

This application claims the benefit of U.S. Provisional Application No. 63/164,009, filed 22 Mar. 2021, the entire disclosure of which is hereby incorporated by reference herein.

### TECHNICAL FIELD

The present disclosure relates to the use of porphyrin structures, such as porphyrin nanoparticles, as antimicrobial agents for broad spectrum antibacterial, antifungal and/or antiviral agents. The porphyrin structures are useful for reducing or inhibiting growth of microbes over an extended period of time after application.

### BACKGROUND

Microbes are omnipresent in the environment and some have negative health consequences. Contamination of a surface from infectious microbes can occur through air and through transmission by contact from persons, animals, objects, etc. Cleaning with a disinfectant can reduce the amount of microbes on contaminated surfaces and can thus decrease risk of infection and transmission from such surfaces. However, many microbes have become resistant to common disinfectants and many disinfectants do not provide for extended periods of disinfection, e.g., less than 24 hours, and can be consumed as part of their activity.

A continuing need exists to develop antimicrobial agents particularly those that are non-toxic and have a broad spectrum antibacterial, antifungal and antiviral effect. There is a further need to develop antimicrobial agents and formulations containing such agents that can reduce or inhibit growth of microbes over an extended period of time.

### SUMMARY OF THE DISCLOSURE

An advantage of the present disclosure is using porphyrin structures, such as porphyrin nanostructures and nanoparticles, as antimicrobial agents particularly those that are non-toxic and have a broad spectrum antibacterial, antifungal and antiviral effect. Additional advantages of the present disclosure include antimicrobial formulations including porphyrin nanoparticles having antimicrobial activity (with and without visible light) over an extended period of time and at low formulation concentrations and applied to yield low surface densities.

These and other advantages are satisfied, at least in part, by methods of reducing or inhibiting the growth of a microbe by applying a formulation including porphyrin nanoparticles to a surface to impart a residue of porphyrin nanoparticles on the surface to reduce or inhibit the growth of the microbe (with and without visible light) on the surface. The methods include applying such a formulation to reduce or inhibit growth of one or more viruses, e.g., SARS, SARS-CoV-2, an Influenza, a Rhinovirus and/or a Norovirus. Other methods include reducing or inhibiting growth of a broad spectrum of infectious bacteria microbes by applying such a formulation to reduce or inhibit the growth of the at least two infectious microbes, wherein at least one of the infectious microbes is a multi-drug resistant bacteria, e.g., a multi-drug resistant (MDR) Escherichia coli or methicillin-resistant S. aureus.

Another aspect of the present disclosure includes methods of reducing or inhibiting growth of a microbe over an extended period of time by applying a formulation including porphyrin nanoparticles to a surface to impart a residue of porphyrin nanoparticles on the surface to reduce or inhibit the growth of the microbe (with and without visible light) on the surface for an extended period of time. Such extended periods of time include a period of at least 24 hours, e.g., at least 2 days, at least one week, etc.

Another aspect of the present disclosure includes an antimicrobial formulation comprising (i) a fast acting disinfectant and (ii) porphyrin nanoparticles, wherein application of the formulation to a surface imparts a residue of porphyrin nanoparticles on the surface having antimicrobial activity. The fast acting disinfectant can include one or more quaternary ammonium compounds.

Embodiments of the present disclosure include one or more of the following features individually or combined. For example, the formulation can include the porphyrin nanoparticles at a concentration from about 10⁻⁶ μg/ml up to about 1 mg/ml, e.g., from about 10⁻⁶ μg/ml up to about 0.1 mg/ml. In other embodiments, the residue of the porphyrin nanoparticles applied to a surface can have an average surface density of from about 10⁻⁶ μg/cm² up to about 1 mg/cm², e.g., about 10⁻⁶ μg/cm² up to about 0.1 mg/cm². Formulations of the present disclosure can be applied to microbe contaminated surfaces and/or surfaces susceptible to contamination, e.g., by contact with infectious microbes through persons, animals, or objects. In some embodiments, the antimicrobial formulation can include a fast acting disinfectant which is different from the porphyrin nanoparticles. In other embodiments, the antimicrobial formulations can include iron-based nanoparticles, zinc-based nanoparticles, silver-based nanoparticles, and/or fluorinated-based porphyrin nanoparticles.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF THE DRAWINGS

Reference is made to the attached drawings, wherein elements having the same reference numeral designations represent similar elements throughout and wherein:

FIG. 1: Bacteria colonization on control (uncoated) surfaces (top) and surfaces coated with FeTCP (0.001 mg/ml) after 2 weeks in visible light (bottom) showing complete bacteria inhibition on the FeTCP coated surfaces (bottom). Data=mean+/−SEM; N=3. Bacteria were seeded at 10⁶ CFU on each sample.

FIG. 2: Virus attachment on control (uncoated) surfaces (top) and surfaces coated with FeTCP (0.001 mg/ml) after 3 weeks in visible light (bottom) showing complete virus attachment inhibition on the FeTCP coated surfaces (bottom). Data=mean+/−SEM; N=3. Viruses were seeded at 10⁶ copies on each sample.

FIG. 3: Bacteria colonization on control (uncoated) surfaces (top) and surfaces coated with either ZnTPyP, AgTPP, F-DCP, or FeTCP (0.001 mg/ml) after 10 minutes in the light (bottom) showing significant or complete bacteria inhibition. Data=mean+/−SEM; N=3. Bacteria were seeded at 10⁶ CFU on each sample.

FIG. 4: Bacteria colonization on control (uncoated) surfaces (top) and surfaces coated with either ZnTPyP, AgTPP, F-DCP or FeTCP (0.001 mg/ml) after 10 minutes in the dark (i.e., no visible light) (bottom) showing significant or complete bacteria inhibition (bottom). Data=mean+/−SEM; N=3. Bacteria were seeded at 10⁶ CFU on each sample.

FIGS. 5A, 5B, 5C and 5D: Bacteria colonization on surfaces coated with ZnTPyP at various concentrations (1 mg/ml to 0.00001 mg/ml) after 4 hours in the dark (i.e., no visible light) showing significant. Data=mean+/−SEM; N=3. Bacteria were seeded at 10⁶ CFU on each sample.

### DETAILED DESCRIPTION OF THE DISCLOSURE

The present disclosure relates to the use of porphyrin structures, e.g., porphyrin nanoparticles, as antimicrobial agents particularly those that are non-toxic and have a broad spectrum antibacterial, antifungal and antiviral effect. As used herein, “antimicrobial” refers to both microbicidal and microbiostatic properties. That is, the term includes microbe killing, leading to a reduction in number of microbes, and/or retarding effect of microbial growth, wherein numbers may remain more or less constant (but nonetheless allowing for slight increase or decrease). As used herein, the terms microbe or microorganism refer to bacteria, fungi, protozoa, and viruses. The porphyrin nanoparticles of the present disclosure have a broad spectrum antibacterial, antifungal and antiviral activity under both light and dark conditions and over extended periods of time.

The porphyrin nanoparticles can range in average size from no more than about 10 microns, such as no more than about 1 micron and can range in average size from about 10 nm to about 10 microns. As used herein a porphyrin nanostructure has an average size in any dimension of less than 1,000 nm. Further, the porphyrin nanoparticles can be of various geometries, spherical, fibrous, cuboidal, pyramidal, etc. The porphyrin structures of the present disclosure can be easily applied to any surface to inhibit and deactivate bacteria as well as viruses. Many such porphyrin structures are non-toxic and non-damaging to the underlying surface.

In particular, the disclosure relates to porphyrin nanoparticles for use as antimicrobial agents. Syntheses of porphyrin structures were reported in U.S. Pat. No. 8,871,926, which is incorporated herein in its entirety by reference. Such porphyrin structures were disclosed to display photocatalytic behavior, photovoltaic behavior, fluorescence, and gas storage capability. See also Wang et al., ACS Nano 2018, 12, 4, 3796-3803, disclosing that under light irradiation, ZnTPyP@NO nanoparticles release highly reactive peroxynitrite molecules that exhibit enhanced antibacterial photodynamic therapy (APDT) activity.

The porphyrin nanoparticles according to the present disclosure, however, do not require nitric oxide and have antimicrobial activity under visible light and without visible light (i.e., in the dark) for prolonged periods of time (up to but not limited to 6 weeks) at low concentrations (e.g., less than 1 μg/cm²). In one aspect of the present disclosure, the porphyrin nanoparticles are not combined with nitric oxide (NOx) to form NOx absorbed particles, e.g., ZnTPyP@NO nanoparticles, apart from which may occur due to natural presence of NO under atmospheric pressure. Hence, in some embodiments, the porphyrin nanoparticles of the present disclosure are significantly free of nitric oxide, e.g., less than 1 wt %, if any NOx absorbed on the nanoparticles.

The porphyrin structures can be prepared through surfactant-assisted non-covalent interactions. See U.S. Pat. No. 8,871,926. Such processes allow control of the dimension and morphology of the porphyrin structures. This process creates nano dimensional porphyrins structures. In general, the process includes mixing a stock porphyrin solution with an acidic solution, e.g. mixing with a hydrogen chloride solution, or alkaline solution and stirring the mixture for a period of time, e.g., 30 min. This mixture is then added into a continuously stirred aqueous solution of a surfactant such as cetyl trimethylammonium bromide (CTAB). The surfactant solution can be acidic or alkaline depending on the stock metal ligand porphyrin mixture. The stock metal ligand porphyrin mixture and surfactant solutions are mixed for a period of time until porphyrin nanostructure form. The porphyrin structures can be separated from the mixture by centrifugation and washed to remove the surfactant. After the porphyrin nanoparticles are separated from the mixture, they can be sterilized by UV light or other high intensity light. It is believed that sterilization facilitates the antimicrobial activity of the porphyrin nanoparticles.

A wide variety of porphyrin nanoparticles can be made according to the process starting from porphyrins having a porphine inner core and at least two pendant metal-coordinating (chelating) moieties. In certain aspects, preferred porphyrin nanoparticles include those prepared from an iron-based porphyrin such as iron(III) meso-tetra(4-pyridyl) porphine chloride (Fe(III)-TPyP or FeTPyP), iron (III) meso-tetra (4-carboxyphenyl) porphine chloride (Fe(III)-TCP or FeTCP), iron (III) meso-tetra (4-sulfonatophenyl) porphine chloride (acid form) (Fe(III)-TSP), etc., zinc-based porphyrins such as zinc 5,10,15,20 tetra (4-pyridyl)-porphine (ZnTPyP), zinc (II) meso-trans-di(4-pyridyl) diphenyl porphine (Zn-DPyDPP), zinc (II) meso-tetra (2-pyridyl) porphine (Zn-T2PyP), zinc (II) meso-tetra (3-pyridyl) porphine (Zn-T3PyP), etc., silver-based porphyrins such as silver (II) meso-tetra(4-pyridyl) porphine (AgTPyP), silver (II) meso-tetraphenyl porphine (AgTPP), etc., a fluorinated porphyrin such as 5,15-di(Pentafluorophenyl)-10,20-di(2-carboxyphenyl) (F-DCP), a manganese (III) octaethyl chloride nanoporphyrin, a platinum (ii) octaethyl nanoporphyrin, a platinum (II) meso-tetra(pentafluorophenyl) nanoporphyrin, a 5,15-di(4-aminophenyl)-10,20-diphenyl nanoporphyrin, a copper (II) octaethyl nanoporphyrin, a palladium (II) meso-tetraphenyl tetrabenzo nanoporphyrin, or a magnesium (II) octaethyl nanoporphyin. The iron-based porphyrins, zinc-based porphyrins, silver-based porphyrins, fluorine-based porphyrins, manganese-based porphyrins, platinum-based porphyrins, amino-based porphyrins, copper-based porphyrins, palladium-based porphyrins, and magnesium-based porphyrin can be formed into iron-, zinc-, silver-, fluorinated-, manganese-, platinum-, amino-, copper-, palladium-, and magnesium-based porphyrin nanoparticles according to the present disclosure. We have found that the iron, zinc, silver, fluorine, and magnesium based porphyrin nanoparticles have high antimicrobial activity, both in light and dark, and over extended periods of time, e.g., for a period of at least 24 hours, 2 days, 1-2 weeks, etc., at very low concentrations, e.g. less than 1 μg/cm².

Other porphyrins can be used to prepare nanoparticles but such other porphyrins have limited usefulness which include 5,10,15,20-tetra (4-pyridyl)-21H,23H-porphine, tin (IV) meso-tetra (4-pyridyl) porphine dichloride(Sn-TPyP), titanium oxide meso-tetra (4-pyridyl) porphine (TiO-TPyP), vanadium oxide meso-tetra (4-pyridyl) porphine (VO-TPyP), cobalt (III) meso-tetra (4-pyridyl) porphine chloride (Co-TPyP), manganese (III) meso-tetra (4-pyridyl) porphine chloride, nickel (II) meso-tetra (4-pyridyl) porphine (Ni-TPyP), tetra (4-carboxyphenyl) porphine (TCP), copper (II) meso-tetra (4-carboxyphenyl) porphine (Cu-TCP), palladium (II) meso-tetra (4-carboxyphenyl) porphine (Pd-TCP), 5,10,15,20-tetrakis (4-hydroxyphenyl)-21H,23H-porphine (THP), and 5, 15-di(4-pyridyl)-10,20-diphenylporphyrin (DPyDPP).

Starting from a porphyrin solution, porphyrin nanoparticles can be produced which exhibit unique optical and electrical properties that are advantageous for effective antimicrobial activity including efficient antibacterial and antiviral activity, with enhanced properties due to the collective behavior resulting from their assembly. Through kinetic control, the process readily allows for fine-tuning of the nanostructure, dimension, and function on multiple length scales.

The porphyrin particles can range in size from no more than an average size of about 10 microns, such as no more than about 1 micron on average and can range from about 10 nm to about 10 microns on average.

In an aspect of the present disclosure, porphyrin nanoparticles are combined with a medium to form an antimicrobial formulation. The porphyrin nanoparticles are typically dispersed in the medium. As such the antimicrobial formulation includes one or more porphyrin nanoparticles and the medium. Useful mediums can include, for example, water, aqueous solutions including as NaOH solutions, salt solutions such as NaCl solutions, acidic solutions such as HCl solutions, etc., lower alcohols such as ethanol, isopropyl alcohol, a butanol or propanol or mixtures of water and alcohols together with additional ingredients. The one or more porphyrin nanoparticles can be included in the formulation at a sufficient concentration for antimicrobial activity when the formulation is applied to a surface such as from about 0.00000001 mg/ml (10⁻⁶ μg/ml) to about 1 mg/ml, such as from about 10⁻⁶ μg/ml, 0.0000001 mg/ml, 0.000001 mg/ml, 0.00001 mg/ml, 0.0001 mg/ml, etc. up to about 0.001 mg/ml, e.g., up to about 0.01 mg/ml, 0.1 mg/ml, 1 mg/ml.

In another aspect, the antimicrobial formulations of the present disclosure can include one or more disinfectants that are different than a porphyrin nanoparticle. Such additional disinfectant preferably includes a relatively fact acting, relatively short lived disinfectant such as one or more quaternary ammonium compounds, e.g., benzalkonium chloride, also known as alkyldimethylbenzylammonium chloride such as dimethyl benzyl ammonium chloride, didecyl dimethyl ammonium chloride, octyl decyl dimethyl ammonium chloride, dioctyl dimethyl ammonium chloride, etc.

The formulation can also include additional optional components such as one or more of a binder, such as an polyoxazoline, a surfactant, such as nonionic, cationic, anionic, zwitterionic surfactants, e.g., sulphonic acid, alkylbenzene sulphonates, sodium laureth sulfate, cetrimonium bromide or CTAB, alkylethoxylate C12-14 or 7 EO, alkylpolyglycoside (C9-C11), C9-11 pareth-6, alkyl dimethyl amine oxide, coco amido propyl betaine, a colorant, a fragrance, a chelating agent, buffering agent, foaming agent, cleaner, additives, e.g., chelants methyl glycinediacetic acid or MGDA, ethylenediaminetetraacetic acid or EDTA, benzalkonium chloride or BKC, monoethanoloamine, propylene glycol phenyl ether or propylene glycol butyl ether among other ingredients.

The antimicrobial formulation of the present disclosure can be applied to a surface by spraying, rolling, fogging, wiping, among other methods. The formulations can be applied to a variety of surface materials such as metals, glasses, textiles, wood, plastics, fabrics, etc. and on a variety of devices and objects susceptible to microbe contamination through air-born contamination or by contact with persons, animals, objects, etc. Such infectious microbial susceptible surfaces include, for example, personal electronic devices, door handles, countertops household appliances, bathroom fixtures, personal apparel, clothes, cupholders, seats, dashboards, and air filtration systems, etc.

Once dried, antimicrobial formulations of the present disclosure leave a residual protective residue of porphyrin nanoparticles having antimicrobial activity against a broad spectrum of bacteria, viruses and fungi over an extended period of time under both exposure to visible light and without exposure to visible light. The residue of the porphyrin nanoparticles on the surface has an average surface density of from about 10⁻⁶ μg/cm² up to about 10 mg/cm². For example, the porphyrin nanoparticles can be applied from an antimicrobial formulation having a concentration of from about 10⁻⁶ μg/ml up to about 1 mg/ml to a surface to imparts a residue of porphyrin nanoparticles having a surface density of from about 10⁻⁶ μg/cm², 0.0000001 mg/cm², 0.000001 mg/cm², 0.00001 mg/cm², 0.0001 mg/cm², etc. up to about 0.001 mg/cm², e.g., up to about 0.01 mg/cm², 0.1 mg/cm², 1 mg/cm², etc.

Once applied to a surface, antimicrobial formulations of the present disclosure act as a surface disinfectant to kill, reduce, and/or inhibit growth of infectious microbes present on or contacting the surface over an extended period of time under both exposure to visible light and without exposure to visible light. Such infectious microbes include, for example, gram-positive bacteria, gram-negative bacteria and combinations thereof, e.g., Escherichia coli, multi-drug resistant (MDR) bacteria such as MDR Escherichia coli, methicillin-resistant S. aureus (MRSA), S. aureus, Staphylococcus epidermidis, and Pseudomonas aeuroginosa, etc., infection fungi such as Candida albicans, infectious viruses such as respitory syricytial virus, SARS-COV-2, Influenzae such as Influenza A and B, Rhinovirus, Norovirus, HIV, MERS, etc.

In an aspect of the present disclosure application of antimicrobial formulation to a surface imparts a residue of porphyrin nanoparticles on the surface having antimicrobial activity to reduce microbes or inhibit growth of microbes (with and without visible light) on the surface for a period of at least 24 hours after application of the formulation, e.g., at least 2 days, one week, two weeks, three weeks, four weeks, etc. after applying porphyrin nanoparticles to the surface.

Embodiments of the present application include applying a formulation including porphyrin nanoparticles to a surface to reduce or inhibit the growth of microbes. Such surfaces can have infectious microbes or come in contact with such infectious microbes. The microbes can be infectious viruses, bacteria, fungi and having or contacting at least two infectious microbes including at least one multi-drug resistant bacteria, e.g., MDR Escherichia coli or methicillin-resistant S. aureus.

The antimicrobial formulations of the present application including iron, zinc, silver and fluorine based porphyrin nanoparticles at concentrations ranging from about 10⁶ μg/ml to about 1 mg/ml demonstrate high antiviral activity on a variety of surface materials and objects over extended periods of time.

Table 1 below show results of virus attachment on surfaces coated with ZnTPyP, AgTPP, F-DCP or FeTCP (0.0001 mg/ml) at various exposure periods in visible light and without light (Dark). Data=mean+/−SEM; N=3. Viruses were seeded at 10⁶ copies on each sample. As shown by Table 1, the antimicrobial formulations of the present disclosure applied to a variety of surfaces exhibit significant or complete virus attachment inhibition on coated surfaces over extended periods of time.

TABLE 1
Antiviral Activity of Formulations

| | | | | Log Reduction | | | |
|---|---|---|---|---|---|---|---|
| | | Exposure | | SARS- | Influenza | Rhino- | Noro- |
| Porphyrin | Conditions | Time | Material | CoV-2 | A | virus | virus |
| ZnTPyP | Light | 10 | Aluminum | 0.60 | 6.00 | 0.60 | 0.48 |
| | | minutes | Granite Countertop | 0.95 | 6.00 | 6.00 | 0.60 |
| | | | Polypropylene | 6.00 | 6.00 | 6.00 | 6.00 |
| | | | Carpet | 6.00 | 6.00 | 6.00 | 0.95 |
| ZnTPyP | Dark | 10 | Aluminum | 0.18 | 0.78 | 0.18 | 0.18 |
| | | minutes | Granite Countertop | 0.90 | 0.90 | 0.78 | 0.48 |
| | | | Polypropylene | 0.85 | 0.78 | 0.85 | 0.60 |
| | | | Carpet | 0.95 | 0.95 | 0.95 | 0.95 |
| ZnTPyP | Light | 1 | Aluminum | 6.00 | 6.00 | 0.60 | 0.48 |
| | | week | Granite Countertop | 0.95 | 6.00 | 6.00 | 0.60 |
| | | | Polypropylene | 6.00 | 6.00 | 0.60 | 0.70 |
| | | | Carpet | 0.95 | 6.00 | 6.00 | 0.95 |
| ZnTPyP | Dark | 1 | Aluminum | 0.00 | 0.78 | 0.18 | 0.18 |
| | | week | Granite Countertop | 0.60 | 0.60 | 0.78 | 0.48 |
| | | | Polypropylene | 0.85 | 0.48 | 0.54 | 0.30 |
| | | | Carpet | 0.95 | 0.95 | 0.95 | 0.48 |
| ZnTPyP | Light | 2 | Aluminum | 6.00 | 6.00 | 0.60 | 0.48 |
| | | weeks | Granite Countertop | 0.95 | 6.00 | 6.00 | 0.60 |
| | | | Polypropylene | 6.00 | 6.00 | 0.60 | 0.70 |
| | | | Carpet | 0.95 | 6.00 | 6.00 | 0.95 |
| ZnTPyP | Dark | 2 | Aluminum | 0.48 | 0.48 | 0.00 | 0.00 |
| | | weeks | Granite Countertop | 0.90 | 0.90 | 0.78 | 0.48 |
| | | | Polypropylene | 0.54 | 0.30 | 0.37 | 0.12 |
| | | | Carpet | 0.65 | 0.65 | 0.65 | 0.48 |
| ZnTPyP | Light | 3 | Aluminum | 0.60 | 0.70 | 0.30 | 0.18 |
| | | weeks | Granite Countertop | 6.00 | 0.95 | 6.00 | 0.60 |
| | | | Polypropylene | 6.00 | 6.00 | 0.60 | 0.40 |
| | | | Carpet | 6.00 | 0.95 | 0.95 | 0.95 |
| ZnTPyP | Dark | 3 | Aluminum | 0.00 | 0.30 | 0.12 | 0.00 |
| | | weeks | Granite Countertop | 0.60 | 0.60 | 0.48 | 0.18 |
| | | | Polypropylene | 0.37 | 0.30 | 0.24 | 0.00 |
| | | | Carpet | 0.65 | 0.65 | 0.48 | 0.48 |
| AgTPP | Light | 10 | Aluminum | 6.00 | 6.00 | 0.60 | 0.48 |
| | | minutes | Granite Countertop | 6.00 | 0.95 | 6.00 | 6.00 |
| | | | Polypropylene | 6.00 | 6.00 | 6.00 | 6.00 |
| | | | Carpet | 6.00 | 0.95 | 6.00 | 6.00 |
| AgTPP | Dark | 10 | Aluminum | 0.48 | 0.78 | 0.48 | 0.48 |
| | | minutes | Granite Countertop | 6.00 | 0.90 | 6.00 | 6.00 |
| | | | Polypropylene | 0.85 | 6.00 | 0.85 | 0.60 |
| | | | Carpet | 6.00 | 0.95 | 6.00 | 6.00 |
| AgTPP | Light | 1 | Aluminum | 6.00 | 0.70 | 0.60 | 0.48 |
| | | week | Granite Countertop | 0.95 | 6.00 | 6.00 | 6.00 |
| | | | Polypropylene | 6.00 | 6.00 | 0.90 | 0.70 |
| | | | Carpet | 6.00 | 0.95 | 6.00 | 6.00 |
| AgTPP | Dark | 1 | Aluminum | 0.48 | 0.78 | 0.48 | 0.48 |
| | | week | Granite Countertop | 0.90 | 0.90 | 0.78 | 0.48 |
| | | | Polypropylene | 0.54 | 0.78 | 0.85 | 0.30 |
| | | | Carpet | 0.95 | 0.95 | 0.95 | 0.95 |
| AgTPP | Light | 2 | Aluminum | 6.00 | 0.70 | 0.60 | 0.48 |
| | | weeks | Granite Countertop | 0.95 | 6.00 | 6.00 | 6.00 |
| | | | Polypropylene | 6.00 | 0.85 | 0.90 | 0.70 |
| | | | Carpet | 6.00 | 6.00 | 6.00 | 6.00 |
| AgTPP | Dark | 2 | Aluminum | 0.18 | 0.78 | 0.18 | 0.18 |
| | | weeks | Granite Countertop | 0.60 | 0.43 | 0.78 | 0.48 |
| | | | Polypropylene | 0.54 | 0.48 | 0.54 | 0.12 |
| | | | Carpet | 0.65 | 6.00 | 0.95 | 0.65 |
| AgTPP | Light | 3 | Aluminum | 6.00 | 0.70 | 0.60 | 0.18 |
| | | weeks | Granite Countertop | 0.95 | 0.95 | 0.85 | 0.60 |
| | | | Polypropylene | 0.90 | 6.00 | 0.90 | 0.40 |
| | | | Carpet | 0.95 | 0.95 | 0.95 | 0.95 |
| AgTPP | Dark | 3 | Aluminum | 0.00 | 0.30 | 0.18 | 0.00 |
| | | weeks | Granite Countertop | 0.60 | 0.43 | 0.48 | 0.00 |
| | | | Polypropylene | 0.54 | 0.30 | 0.54 | 0.12 |
| | | | Carpet | 0.65 | 0.48 | 0.48 | 0.35 |
| F-DCP | Light | 10 | Aluminum | 6.00 | 6.00 | 0.60 | 0.48 |
| | | minutes | Granite Countertop | 6.00 | 0.95 | 6.00 | 6.00 |
| | | | Polypropylene | 6.00 | 6.00 | 6.00 | 0.70 |
| | | | Carpet | 6.00 | 6.00 | 6.00 | 6.00 |
| F-DCP | Dark | 10 | Aluminum | 0.18 | 6.00 | 0.48 | 0.48 |
| | | minutes | Granite Countertop | 0.90 | 0.90 | 6.00 | 6.00 |
| | | | Polypropylene | 6.00 | 0.48 | 0.85 | 0.60 |
| | | | Carpet | 0.95 | 6.00 | 6.00 | 6.00 |
| F-DCP | Light | 1 | Aluminum | 6.00 | 6.00 | 0.60 | 0.48 |
| | | week | Granite Countertop | 6.00 | 0.95 | 6.00 | 6.00 |
| | | | Polypropylene | 6.00 | 6.00 | 6.00 | 0.70 |
| | | | Carpet | 6.00 | 0.95 | 0.95 | 0.95 |
| F-DCP | Dark | 1 | Aluminum | 0.18 | 0.78 | 0.48 | 0.48 |
| | | week | Granite Countertop | 0.60 | 0.60 | 0.48 | 0.48 |
| | | | Polypropylene | 0.85 | 0.48 | 0.54 | 0.30 |
| | | | Carpet | 0.65 | 6.00 | 6.00 | 0.95 |
| F-DCP | Light | 2 | Aluminum | 6.00 | 6.00 | 0.60 | 0.48 |
| | | weeks | Granite Countertop | 6.00 | 0.95 | 6.00 | 6.00 |
| | | | Polypropylene | 6.00 | 6.00 | 6.00 | 0.70 |
| | | | Carpet | 6.00 | 0.95 | 0.95 | 0.95 |
| F-DCP | Dark | 2 | Aluminum | 0.00 | 0.78 | 0.18 | 0.48 |
| | | weeks | Granite Countertop | 0.60 | 0.43 | 0.78 | 0.18 |
| | | | Polypropylene | 0.37 | 0.48 | 6.00 | 0.30 |
| | | | Carpet | 0.48 | 6.00 | 6.00 | 0.65 |
| F-DCP | Light | 3 | Aluminum | 0.60 | 0.70 | 0.60 | 0.48 |
| | | weeks | Granite Countertop | 6.00 | 0.65 | 0.85 | 6.00 |
| | | | Polypropylene | 0.90 | 6.00 | 0.90 | 0.18 |
| | | | Carpet | 0.95 | 0.95 | 0.95 | 0.95 |
| F-DCP | Dark | 3 | Aluminum | 0.00 | 0.48 | 0.00 | 0.18 |
| | | weeks | Granite Countertop | 0.60 | 0.43 | 0.48 | 0.18 |
| | | | Polypropylene | 0.24 | 0.30 | 0.85 | 0.12 |
| | | | Carpet | 0.35 | 6.00 | 0.65 | 0.65 |
| FeTCP | Light | 10 | Aluminum | 6.00 | 6.00 | 6.00 | 6.00 |
| | | minutes | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 10 | Aluminum | 6.00 | 6.00 | 6.00 | 6.00 |
| | | minutes | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Light | 1 | Aluminum | 6.00 | 6.00 | 6.00 | 6.00 |
| | | week | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 1 | Aluminum | 6.00 | 6.00 | 6.00 | 6.00 |
| | | week | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Light | 2 | Aluminum | 6.00 | 6.00 | 6.00 | 6.00 |
| | | weeks | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 2 | Aluminum | 6.00 | 6.00 | 6.00 | 6.00 |
| | | weeks | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Light | 3 | Aluminum | 6.00 | 6.00 | 6.00 | 6.00 |
| | | weeks | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 3 weeks | Aluminum | 6.00 | 6.00 | 6.00 | 6.00 |
| | | | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |

In addition to high antiviral activity, the antimicrobial formulations of the present application including iron, zinc, silver and fluorine based porphyrin nanoparticles at concentrations ranging from about 10⁻⁶ μg/ml to about 1 mg/ml demonstrate broad spectrum antibacterial activity, including antibacterial activity against multi-drug resistant bacteria, e.g., multi-drug resistant (MDR) Escherichia coli and methicillin-resistant S. aureus. The broad spectrum antibacterial activity of the formulations of the present disclosure are observed on a variety of surface materials and objects and over extended periods of time.

Table 2 below show results from bacteria colonization on surfaces coated with either ZnTPyP, AgTPP, F-DCP or FeTCP (0.0001 mg/ml) at various exposure periods in visible light and without light (Dark). Data=mean+/−SEM; N=3. Bacteria were seeded at 10⁶ CFU on each sample. As shown by Table 2 below, the antimicrobial formulations of the present disclosure applied to a variety of surfaces exhibit significant or complete bacteria inhibition on coated surfaces over extended periods of time.

TABLE 2
Broad Spectrum Antibacterial And MDR Activity of Formulations

| | | | | Log Reduction | | | |
|---|---|---|---|---|---|---|---|
| | | Exposure | | Staph | | E. | MDR |
| Porphyrin | Conditions | Time | Material | aureus | MRSA | coli | E. coli |
| ZnTPyP | Light | 10 | Aluminum | 1.90 | 1.63 | 6.00 | 6.00 |
| | | minutes | Granite Countertop | 1.65 | 6.00 | 1.94 | 6.00 |
| | | | Polypropylene | 1.93 | 6.00 | 1.83 | 1.83 |
| | | | Carpet | 6.00 | 6.00 | 6.00 | 6.00 |
| ZnTPyP | Dark | 10 | Aluminum | 1.00 | 1.12 | 1.22 | 1.44 |
| | | minutes | Granite Countertop | 1.26 | 1.35 | 1.35 | 1.38 |
| | | | Polypropylene | 1.06 | 1.22 | 1.21 | 1.42 |
| | | | Carpet | 2.00 | 1.91 | 1.96 | 1.90 |
| ZnTPyP | Light | 1 | Aluminum | 1.60 | 1.65 | 6.00 | 1.18 |
| | | week | Granite Countertop | 1.67 | 6.00 | 6.00 | 6.00 |
| | | | Polypropylene | 1.95 | 6.00 | 1.59 | 1.51 |
| | | | Carpet | 6.00 | 6.00 | 1.98 | 6.00 |
| ZnTPyP | Dark | 1 | Aluminum | 1.07 | 0.99 | 0.87 | 1.17 |
| | | week | Granite Countertop | 1.19 | 1.18 | 0.99 | 1.21 |
| | | | Polypropylene | 1.00 | 1.00 | 1.05 | 1.29 |
| | | | Carpet | 1.70 | 1.60 | 1.96 | 1.35 |
| ZnTPyP | Light | 2 | Aluminum | 1.60 | 1.64 | 6.00 | 0.70 |
| | | weeks | Granite Countertop | 1.67 | 6.00 | 6.00 | 6.00 |
| | | | Polypropylene | 1.95 | 6.00 | 1.51 | 0.70 |
| | | | Carpet | 6.00 | 6.00 | 1.94 | 6.00 |
| ZnTPyP | Dark | 2 | Aluminum | 1.07 | 0.99 | 0.97 | 0.70 |
| | | weeks | Granite Countertop | 1.18 | 1.17 | 0.98 | 0.52 |
| | | | Polypropylene | 1.00 | 1.00 | 1.04 | 0.40 |
| | | | Carpet | 1.70 | 1.60 | 1.90 | 0.40 |
| ZnTPyP | Light | 3 | Aluminum | 1.30 | 1.18 | 1.18 | 0.70 |
| | | weeks | Granite Countertop | 1.49 | 1.40 | 1.33 | 0.70 |
| | | | Polypropylene | 1.65 | 1.38 | 1.21 | 0.52 |
| | | | Carpet | 2.00 | 1.54 | 1.62 | 0.70 |
| ZnTPyP | Dark | 3 | Aluminum | 0.91 | 1.22 | 0.77 | 0.52 |
| | | weeks | Granite Countertop | 1.12 | 1.05 | 0.90 | 0.40 |
| | | | Polypropylene | 0.99 | 0.85 | 1.05 | 0.40 |
| | | | Carpet | 1.40 | 1.30 | 1.43 | 0.30 |
| AgTPP | Light | 10 | Aluminum | 6.00 | 6.00 | 6.00 | 6.00 |
| | | minutes | Granite Countertop | 6.00 | 6.00 | 1.94 | 1.63 |
| | | | Polypropylene | 6.00 | 6.00 | 1.83 | 6.00 |
| | | | Carpet | 6.00 | 1.85 | 6.00 | 6.00 |
| AgTPP | Dark | 10 | Aluminum | 1.54 | 1.60 | 1.39 | 1.44 |
| | | minutes | Granite Countertop | 1.35 | 6.00 | 1.95 | 6.00 |
| | | | Polypropylene | 1.30 | 1.70 | 1.91 | 1.90 |
| | | | Carpet | 2.00 | 6.00 | 1.96 | 6.00 |
| AgTPP | Light | 1 | Aluminum | 1.90 | 6.00 | 1.48 | 1.48 |
| | | week | Granite Countertop | 6.00 | 6.00 | 1.51 | 6.00 |
| | | | Polypropylene | 6.00 | 6.00 | 6.00 | 1.81 |
| | | | Carpet | 6.00 | 1.54 | 1.93 | 6.00 |
| AgTPP | Dark | 1 | Aluminum | 1.37 | 1.30 | 1.12 | 1.17 |
| | | week | Granite Countertop | 1.27 | 1.65 | 1.65 | 1.38 |
| | | | Polypropylene | 1.20 | 1.22 | 1.60 | 1.59 |
| | | | Carpet | 1.70 | 1.91 | 1.66 | 1.66 |
| AgTPP | Light | 2 | Aluminum | 1.90 | 6.00 | 1.48 | 1.00 |
| | | weeks | Granite Countertop | 6.00 | 6.00 | 1.64 | 6.00 |
| | | | Polypropylene | 6.00 | 6.00 | 6.00 | 1.00 |
| | | | Carpet | 6.00 | 1.56 | 1.94 | 6.00 |
| AgTPP | Dark | 2 | Aluminum | 1.37 | 1.29 | 1.10 | 0.70 |
| | | weeks | Granite Countertop | 1.26 | 1.65 | 1.38 | 0.70 |
| | | | Polypropylene | 1.20 | 1.22 | 1.59 | 0.70 |
| | | | Carpet | 1.70 | 1.91 | 1.60 | 0.70 |
| AgTPP | Light | 3 | Aluminum | 1.60 | 1.35 | 1.18 | 0.70 |
| | | weeks | Granite Countertop | 1.49 | 1.40 | 1.33 | 0.70 |
| | | | Polypropylene | 1.95 | 1.38 | 1.51 | 0.52 |
| | | | Carpet | 1.70 | 1.37 | 1.62 | 0.70 |
| AgTPP | Dark | 3 | Aluminum | 1.26 | 1.09 | 0.87 | 0.52 |
| | | weeks | Granite Countertop | 1.19 | 1.18 | 1.20 | 0.52 |
| | | | Polypropylene | 1.12 | 1.22 | 1.42 | 0.70 |
| | | | Carpet | 1.52 | 1.60 | 1.43 | 0.52 |
| F-DCP | Light | 10 | Aluminum | 6.00 | 6.00 | 6.00 | 6.00 |
| | | minutes | Granite Countertop | 6.00 | 1.70 | 1.94 | 1.63 |
| | | | Polypropylene | 6.00 | 6.00 | 6.00 | 6.00 |
| | | | Carpet | 6.00 | 6.00 | 6.00 | 6.00 |
| F-DCP | Dark | 10 | Aluminum | 1.85 | 6.00 | 1.69 | 6.00 |
| | | minutes | Granite Countertop | 1.26 | 6.00 | 6.00 | 1.69 |
| | | | Polypropylene | 1.60 | 1.70 | 1.91 | 1.90 |
| | | | Carpet | 6.00 | 6.00 | 6.00 | 6.00 |
| F-DCP | Light | 1 | Aluminum | 6.00 | 6.00 | 1.48 | 6.00 |
| | | week | Granite Countertop | 1.97 | 1.70 | 6.00 | 1.51 |
| | | | Polypropylene | 6.00 | 6.00 | 6.00 | 6.00 |
| | | | Carpet | 2.00 | 1.85 | 6.00 | 6.00 |
| F-DCP | Dark | 1 | Aluminum | 1.54 | 1.60 | 1.30 | 1.47 |
| | | week | Granite Countertop | 1.19 | 1.65 | 1.65 | 1.69 |
| | | | Polypropylene | 1.42 | 1.22 | 1.60 | 1.59 |
| | | | Carpet | 1.70 | 1.91 | 1.48 | 1.96 |
| F-DCP | Light | 2 | Aluminum | 6.00 | 6.00 | 1.48 | 6.00 |
| | | weeks | Granite Countertop | 1.97 | 1.70 | 6.00 | 1.00 |
| | | | Polypropylene | 6.00 | 6.00 | 6.00 | 6.00 |
| | | | Carpet | 2.00 | 1.86 | 6.00 | 6.00 |
| F-DCP | Dark | 2 | Aluminum | 1.54 | 1.59 | 1.27 | 1.00 |
| | | weeks | Granite Countertop | 1.26 | 1.65 | 1.38 | 1.00 |
| | | | Polypropylene | 1.42 | 1.22 | 1.59 | 0.70 |
| | | | Carpet | 1.70 | 1.91 | 1.43 | 1.00 |
| F-DCP | Light | 3 | Aluminum | 1.60 | 1.18 | 1.18 | 1.00 |
| | | weeks | Granite Countertop | 1.67 | 1.40 | 1.63 | 0.52 |
| | | | Polypropylene | 1.65 | 1.38 | 1.51 | 0.52 |
| | | | Carpet | 1.70 | 1.37 | 1.92 | 0.70 |
| F-DCP | Dark | 3 | Aluminum | 1.38 | 1.39 | 0.99 | 0.70 |
| | | weeks | Granite Countertop | 1.12 | 1.18 | 1.07 | 0.52 |
| | | | Polypropylene | 1.29 | 1.40 | 1.42 | 0.40 |
| | | | Carpet | 1.52 | 1.60 | 1.30 | 0.52 |
| FeTCP | Light | 10 | Aluminum | 6.00 | 6.00 | 6.00 | 6.00 |
| | | minutes | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 10 | Aluminum | 6.00 | 6.00 | 6.00 | 6.00 |
| | | minutes | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Light | 1 | Aluminum | 6.00 | 6.00 | 6.00 | 6.00 |
| | | week | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 1 | Aluminum | 6.00 | 6.00 | 6.00 | 6.00 |
| | | week | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Light | 2 | Aluminum | 6.00 | 6.00 | 6.00 | 6.00 |
| | | weeks | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 2 | Aluminum | 6.00 | 6.00 | 6.00 | 6.00 |
| | | weeks | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Light | 3 | Aluminum | 6.00 | 6.00 | 6.00 | 6.00 |
| | | weeks | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |
| FeTCP | Dark | 3 | Aluminum | 6.00 | 6.00 | 6.00 | 6.00 |
| | | weeks | Granite Countertop | 6.00 | 6.00 | 6.00 | 6.00 |
| | | | Polypropylene | 6.00 | 6.00 | 6.00 | 6.00 |
| | | | Carpet | 6.00 | 6.00 | 6.00 | 6.00 |

As shown by the data in Tables 1 and 2, antimicrobial formulations of the present application including iron, zinc, silver and fluorine based porphyrin nanoparticles demonstrated high antiviral activity and broad spectrum antibacterial activity over extended periods of time under both light and dark conditions. However, formulations including iron-based porphyrin nanoparticles surprisingly showed superior antimicrobial in both light and dark conditions and superior activity over the longest periods of time.

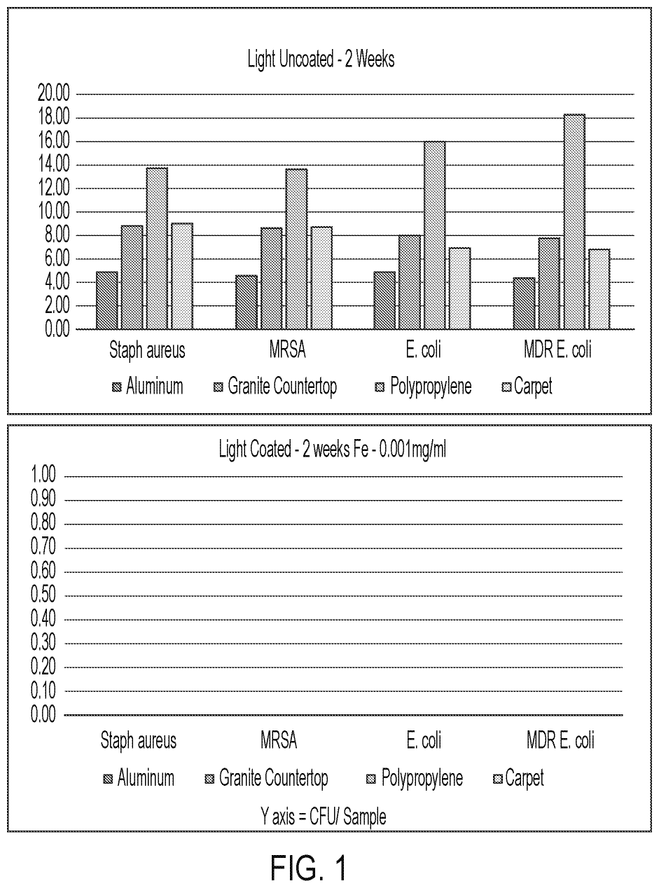

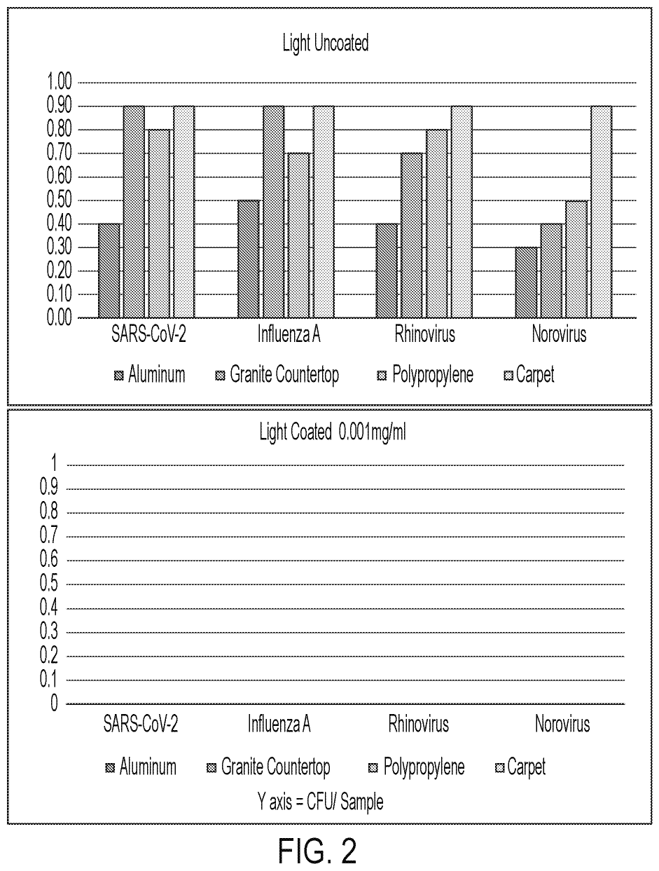

FIGS. 1 and 2 show antibacterial and antiviral activity of a formulation including an iron-based porphyrin (FeTCP). In particular, FIG. 1 shows bacteria colonization on control (uncoated) surfaces (top) and surfaces coated with FeTCP (0.001 mg/ml) after 2 weeks in visible light (bottom). As observed in the control, bacteria increased at the 2 week time period. However, surfaces having the residue formed from FeTCP resulted in complete bacteria inhibition as observed at 2 weeks. FIG. 2 shows virus attachment on control (uncoated) surfaces (top) and surfaces coated with FeTCP (0.001 mg/ml) after 3 weeks in visible light (bottom). As observed in the control in FIG. 2, viral attachment decreased at the 3 week time period. However, surfaces having the residue formed from FeTCP resulted in complete virus attachment inhibition at the 3 week period.

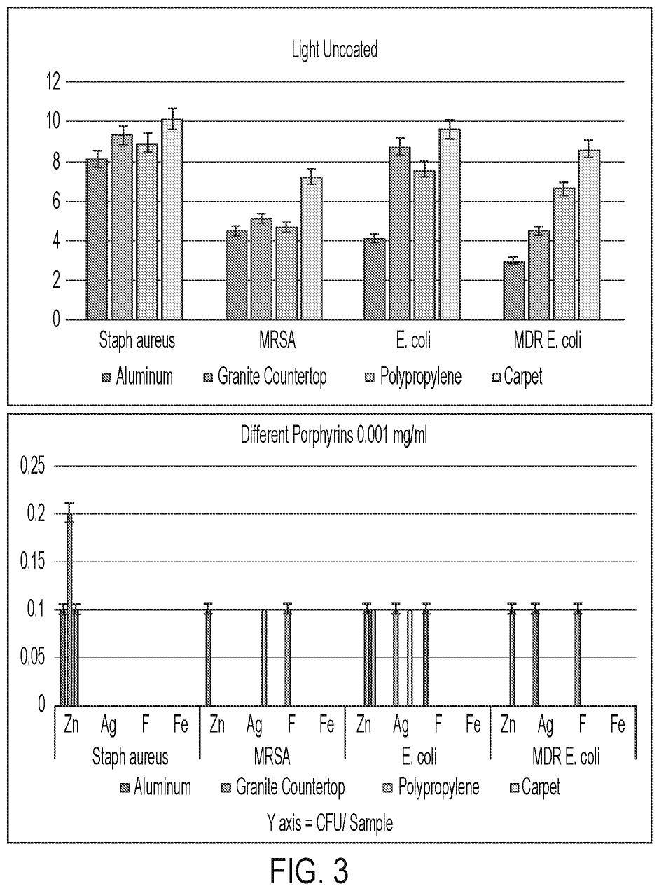

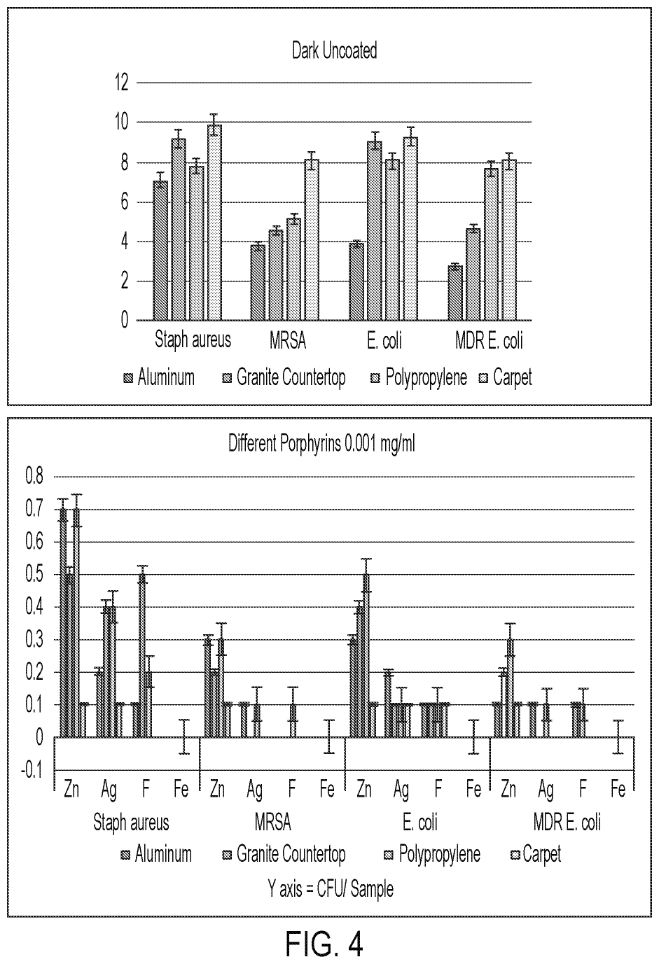

FIGS. 3 and 4 show the relative antimicrobial activity of iron, zinc, silver and fluorine based porphyrin nanoparticles under visible light and without visible light. In particular, FIG. 3 shows bacteria colonization on control (uncoated) surfaces (top) and surfaces coated with either ZnTPyP, AgTPP, F-DCP, or FeTCP (0.001 mg/ml) after 10 minutes in the light (bottom). FIG. 4 shows bacteria colonization on control (uncoated) surfaces (top) and surfaces coated with either ZnTPyP, AgTPP, F-DCP or FeTCP (0.001 mg/ml) after 10 minutes in the dark (i.e., no visible light) (bottom). Both FIGS. 3 and 4 show iron, zinc, silver and fluorine based porphyrin nanoparticles exhibited broad spectrum antibacterial activity (significant or complete bacteria inhibition) under visible light and without visible light. In addition, FIGS. 3 and 4 further demonstrate the superiority of formulations including iron-based porphyrin nanoparticles.

It was further found that antimicrobial formulations having a concentration of porphyrin nanoparticles from about 1 mg/ml to about 0.00001 mg/ml results in significant antimicrobial activity. The data in Table 3 below show that this range of the porphyrin nanoparticles have significant antimicrobial activity and, even though there are orders of magnitude differences in the quantity of the porphyrin nanoparticles, surprisingly the efficacy does not vary as such.

Table 3 below show results from bacteria colonization on surfaces coated with formulations including ZnTPyP at various concentrations in visible light after a 4 hour exposure period. Data=mean+/−SEM; N=3. Bacteria were seeded at 10⁶ CFU on each sample.

TABLE 3
Effects of Concentration Of Porphyrin Nanoparticles in Each Formulation

| | | | Bacterial Log Reduction | | | | | |
|---|---|---|---|---|---|---|---|---|
| | | | Pseudo | Staph | | Staph | E. | MDR |
| Concentration | | Material | aeruginosa | aureus | MRSA | epi | coli | E. coli |
| 0.00001 | mg/ml | Aluminum | 0.40 | 0.15 | 0.22 | 0.15 | 0.30 | 0.40 |
| 0.00001 | mg/ml | Granite Countertop | 0.05 | 0.10 | 0.30 | 0.30 | 0.15 | 0.30 |
| 0.00001 | mg/ml | Polyethylene | 0.30 | 0.05 | 0.15 | 0.30 | 0.30 | 0.10 |
| 0.00001 | mg/ml | PU Coated Desk | 0.15 | 0.00 | 0.15 | 0.40 | 0.05 | 0.22 |
| 0.00001 | mg/ml | Polypropylene | 0.10 | 0.10 | 0.22 | 0.10 | 0.30 | 0.10 |
| 0.00001 | mg/ml | Table cloth | 0.05 | 0.15 | 0.30 | 0.15 | 0.15 | 0.15 |
| 0.00001 | mg/ml | Shirt (Clothes) | 0.10 | 0.10 | 0.22 | 0.30 | 0.30 | 0.10 |
| 0.0001 | mg/ml | Aluminum | 0.15 | 0.52 | 0.70 | 0.52 | 1.00 | 6.00 |
| 0.0001 | mg/ml | Granite Countertop | 0.15 | 0.52 | 6.00 | 6.00 | 1.00 | 1.00 |
| 0.0001 | mg/ml | Polyethylene | 1.00 | 1.00 | 6.00 | 1.00 | 6.00 | 1.00 |
| 0.0001 | mg/ml | PU Coated Desk | 1.00 | 1.00 | 6.00 | 1.00 | 6.00 | 1.00 |
| 0.0001 | mg/ml | Polypropylene | 6.00 | 1.00 | 6.00 | 1.00 | 6.00 | 1.00 |
| 0.0001 | mg/ml | Table cloth | 0.70 | 1.00 | 6.00 | 1.00 | 1.00 | 1.00 |
| 0.0001 | mg/ml | Shirt (Clothes) | 1.00 | 1.00 | 6.00 | 1.00 | 6.00 | 1.00 |
| 0.001 | mg/ml | Aluminum | 1.16 | 1.33 | 1.35 | 1.30 | 6.00 | 1.58 |
| 0.001 | mg/ml | Granite Countertop | 1.11 | 1.28 | 6.00 | 6.00 | 6.00 | 6.00 |
| 0.001 | mg/ml | Polyethylene | 6.00 | 6.00 | 6.00 | 1.91 | 2.01 | 6.00 |
| 0.001 | mg/ml | PU Coated Desk | 6.00 | 6.00 | 6.00 | 6.00 | 6.00 | 6.00 |
| 0.001 | mg/ml | Polypropylene | 6.00 | 6.00 | 6.00 | 6.00 | 6.00 | 6.00 |
| 0.001 | mg/ml | Table cloth | 1.52 | 1.90 | 1.90 | 1.81 | 1.47 | 1.83 |
| 0.001 | mg/ml | Shirt (Clothes) | 1.71 | 1.61 | 1.92 | 1.82 | 1.76 | 1.76 |
| 0.01 | mg/ml | Aluminum | 1.24 | 1.63 | 1.65 | 1.60 | 6.00 | 6.00 |
| 0.01 | mg/ml | Granite Countertop | 1.16 | 1.40 | 6.00 | 6.00 | 6.00 | 6.00 |
| 0.01 | mg/ml | Polyethylene | 6.00 | 6.00 | 1.95 | 1.91 | 6.00 | 6.00 |
| 0.01 | mg/ml | PU Coated Desk | 6.00 | 6.00 | 6.00 | 6.00 | 6.00 | 6.00 |
| 0.01 | mg/ml | Polypropylene | 2.16 | 2.12 | 1.75 | 6.00 | 6.00 | 6.00 |
| 0.01 | mg/ml | Table cloth | 1.69 | 1.90 | 1.90 | 1.81 | 1.47 | 1.83 |
| 0.01 | mg/ml | Shirt (Clothes) | 1.71 | 1.91 | 1.92 | 6.00 | 1.76 | 6.00 |
| 0.1 | mg/ml | Aluminum | 1.34 | 1.63 | 1.65 | 1.60 | 6.00 | 6.00 |
| 0.1 | mg/ml | Granite Countertop | 1.16 | 1.40 | 6.00 | 6.00 | 6.00 | 6.00 |
| 0.1 | mg/ml | Polyethylene | 2.01 | 1.91 | 6.00 | 6.00 | 2.01 | 6.00 |
| 0.1 | mg/ml | PU Coated Desk | 2.05 | 6.00 | 2.00 | 6.00 | 6.00 | 2.19 |
| 0.1 | mg/ml | Polypropylene | 2.16 | 6.00 | 6.00 | 6.00 | 6.00 | 6.00 |
| 0.1 | mg/ml | Table cloth | 1.69 | 6.00 | 6.00 | 6.00 | 1.77 | 6.00 |
| 0.1 | mg/ml | Shirt (Clothes) | 1.71 | 6.00 | 1.92 | 6.00 | 6.00 | 6.00 |
| 1 | mg/ml | Aluminum | 1.24 | 1.63 | 1.65 | 1.60 | 6.00 | 6.00 |
| 1 | mg/ml | Granite Countertop | 1.16 | 1.58 | 6.00 | 6.00 | 6.00 | 6.00 |
| 1 | mg/ml | Polyethylene | 6.00 | 6.00 | 6.00 | 6.00 | 6.00 | 6.00 |
| 1 | mg/ml | PU Coated Desk | 6.00 | 6.00 | 6.00 | 6.00 | 6.00 | 6.00 |
| 1 | mg/ml | Polypropylene | 6.00 | 6.00 | 6.00 | 6.00 | 6.00 | 6.00 |
| 1 | mg/ml | Table cloth | 1.52 | 6.00 | 6.00 | 6.00 | 6.00 | 6.00 |
| 1 | mg/ml | Shirt (Clothes) | 1.71 | 6.00 | 6.00 | 6.00 | 6.00 | 6.00 |

PU in Table 3 refers to polyurethane.

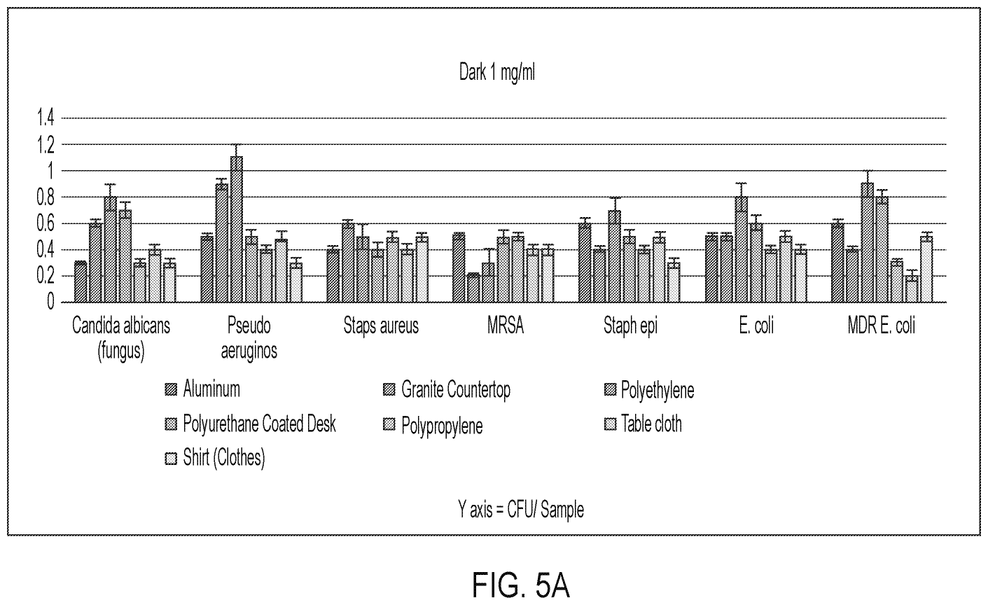

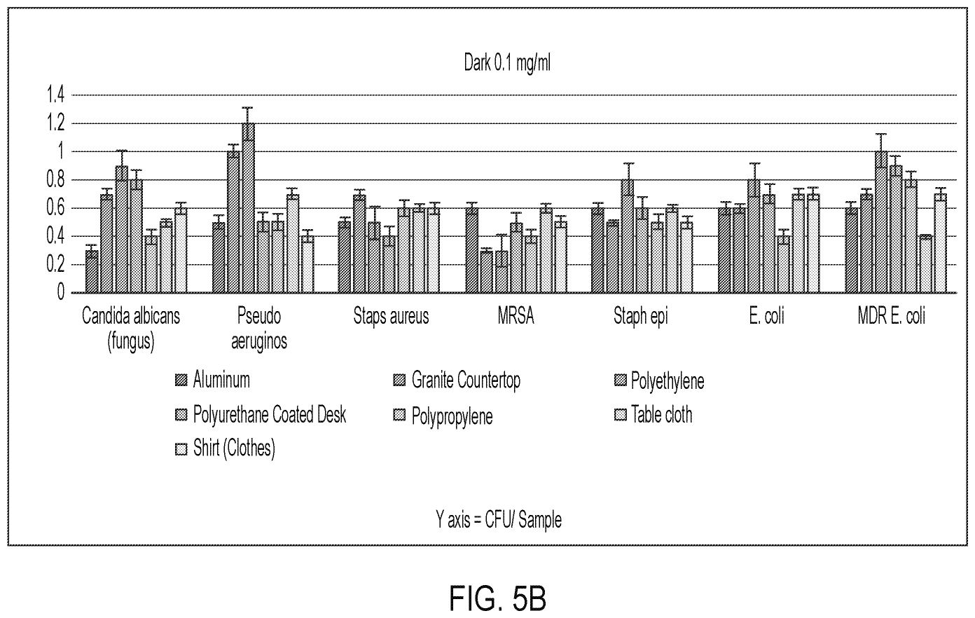

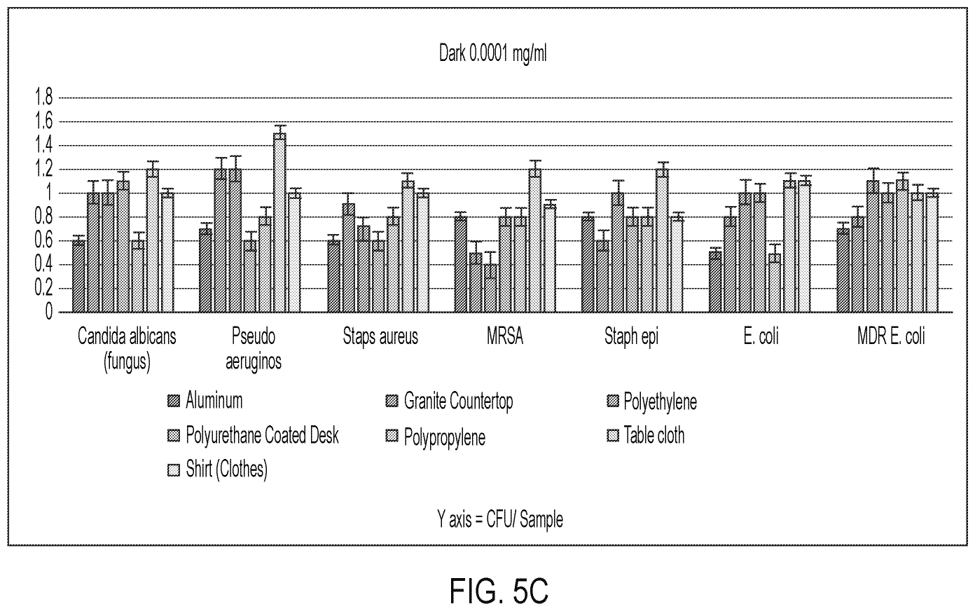

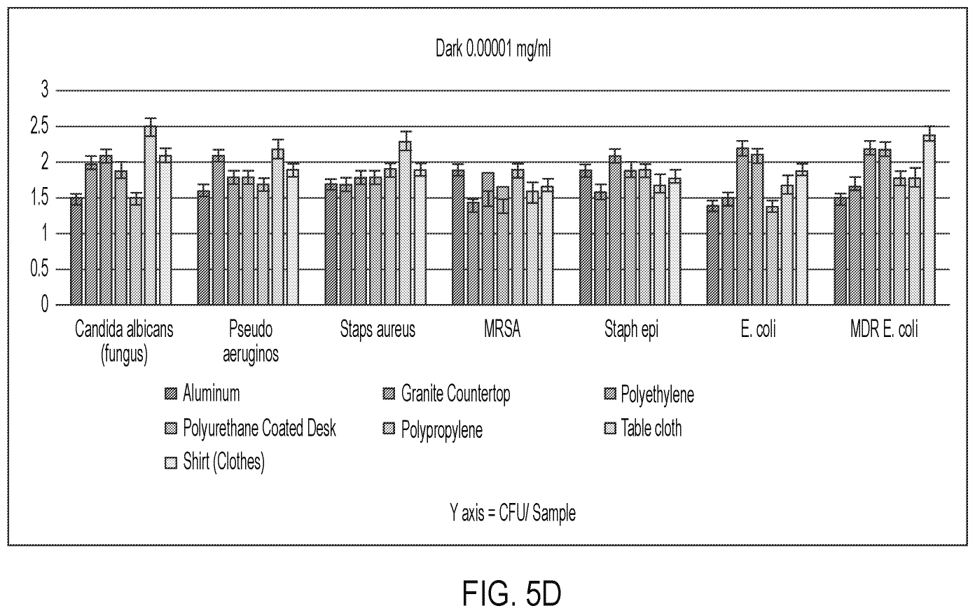

Further, and as shown in FIGS. 5A to 5D, data for porphyrin nanoparticles in the dark within the same concentration range (about 1 mg/ml to about 0.00001 mg/ml) have significant antimicrobial activity. The data for porphyrin nanoparticles in the dark show that the efficacy of the porphyrin nanoparticles does not vary by several orders of magnitude even though the concentration was varied by several orders of magnitude. It is thus believed a concentration range from up to about 1 mg/ml, such as up to about 0.1 mg/ml and no less than about 0.00001 mg/ml, such as no less than about 0.000001 mg/ml of porphyrin nanoparticles in a formulation provides the most benefit of using the materials.

The antimicrobial formulations including porphyrin nanomaterials can be designed to meet the following criteria for disinfectants: when applied, the formulation can have continuous antimicrobial properties, last for more than a few days or weeks before reapplication, not have harmful chemical side effects or reactions, be cost-effective, and can be applied to a variety of surfaces (e.g., porous or non-porous). The nanoparticles can be effective across the antimicrobial spectrum (including viruses, bacteria) and these biocidal effects will last for an extended period of time, e.g., in excess of 24 hour such as two or more days, over one week, two weeks, three, weeks, etc. This usage could become a significant agent to fight the spread of COVID-19 and future viruses. Powered by both natural and artificial light, the nanomaterials are believed to have at least two modes of action. The first is an oxidation mode in which the nanomaterials produce Reactive Oxygen Species (ROS) including peroxynitrites, hydrogen peroxide, and others which have been shown to be highly effective against viruses and bacteria. The second is that when exposed to infrared radiation (such as near infrared radiation), the nanomaterials can absorb the radiation to heat viruses and bacteria up to 93° C. and at the nanoscale.

In another aspect of the present disclosure, antimicrobial formulations of the present disclosure can further include one or more other disinfectants such as one or more quaternary ammonium compounds, e.g., one or more benzalkonium chloride. In fact, it was surprisingly found that the porphyrin nanoparticles of the present disclosure do not appear to deactivate such quaternary ammonium disinfectants and can even be included in existing disinfectant formulations to extend the antimicrobial activity of exiting disinfectant formulations, e.g., Lysol, Microban, Honest. As an example, porphyrin nanoparticles of the present disclosure were added to a Lysol formulation as provided in the Examples below.

Table 4 provides data showing bacteria colonization on control (Lysol only) surfaces and surfaces coated with a formulation including ZnTPyP (0.001 mg/ml) added to Lysol for after 1 week in the light and dark. As shown in Table 4 below, one week after applying a Lysol only formulation to a surface resulted in bacterial growth (negative log reduction value) under both light and dark conditions. However, when porphyrin nanoparticles (ZnTPyP) were added to the same Lysol formulation, the Lysol plus ZnTPyP formulation applied to surfaces after one week exhibited significant broad spectrum bacteria inhibition under both light and dark conditions for a variety of surface materials even for multidrug resistant bacteria.

TABLE 4
Effects of Porphyrin Nanoparticles in Formulations With Other Disinfectants

| | | Bacteria Log Reduction | | | | | |
|---|---|---|---|---|---|---|---|
| | | Pseudo | Staph | | Staph | E. | MDR |
| Condition | Material | aeruginosa | aureus | MRSA | epi | coli | E. coli |
| Dark - Lysol | Aluminum | −0.93 | −0.62 | −0.71 | −0.69 | −0.67 | −0.67 |
| Only | | | | | | | |
| Dark - Lysol | Granite | −1.03 | −0.89 | −0.9 | −0.74 | −0.85 | −0.9 |
| Only | Countertop | | | | | | |
| Dark - Lysol | PE | −0.94 | −0.99 | −0.93 | −0.9 | −0.97 | −1.06 |
| Only | | | | | | | |
| Dark - Lysol | PU Coated | −1.06 | −0.96 | −0.91 | −1 | −1.08 | −1.14 |
| Only | Desk | | | | | | |
| Dark - Lysol | PP | −1.06 | −1.05 | −1.05 | −1.05 | −1.11 | −1.11 |
| Only | | | | | | | |
| Dark - Lysol | Table cloth | −0.98 | −0.9 | −0.92 | −0.86 | −0.77 | −0.84 |
| Only | | | | | | | |
| Dark - Lysol | Shirt | −0.96 | −0.95 | −0.9 | −0.92 | −0.79 | −0.81 |
| Only | (Clothes) | | | | | | |
| Dark - Lysol + | Aluminum | 6.00 | 6.00 | 6.00 | 6.00 | 1.00 | 0.70 |
| ZnTPyP | | | | | | | |
| Dark - Lysol + | Granite | 1.00 | 1.00 | 1.00 | 6.00 | 6.00 | 6.00 |
| ZnTPyP | Countertop | | | | | | |
| Dark - Lysol + | PE | 1.00 | 6.00 | 6.00 | 6.00 | 6.00 | 1.00 |
| ZnTPyP | | | | | | | |
| Dark - Lysol + | PU Coated | 6.00 | 1.00 | 0.70 | 6.00 | 1.00 | 6.00 |
| ZnTPyP | Desk | | | | | | |
| Dark - Lysol + | PP | 1.00 | 6.00 | 6.00 | 6.00 | 6.00 | 1.00 |
| ZnTPyP | | | | | | | |
| Dark - Lysol + | Table cloth | 6.00 | 6.00 | 1.00 | 6.00 | 1.00 | 6.00 |
| ZnTPyP | | | | | | | |
| Dark - Lysol + | Shirt | 1.00 | 6.00 | 6.00 | 1.00 | 6.00 | 1.00 |
| ZnTPyP | (Clothes) | | | | | | |
| Light - Lysol | Aluminum | −0.94 | −0.59 | −0.68 | −0.67 | −0.65 | −0.69 |
| Only | | | | | | | |
| Light - Lysol | Granite | −1.04 | −0.89 | −0.89 | −0.73 | −0.83 | −0.91 |
| Only | Countertop | | | | | | |
| Light - Lysol | PE | −0.94 | −1 | −0.94 | −0.91 | −0.97 | −1.06 |
| Only | | | | | | | |
| Light - Lysol | PU Coated | −1.09 | −0.96 | −0.95 | −1.04 | −1.10 | −1.16 |
| Only | Desk | | | | | | |
| Light - Lysol | PP | −1.1 | −1.09 | −1.06 | −1.06 | −1.14 | −1.14 |
| Only | | | | | | | |
| Light - Lysol | Table cloth | −0.98 | −0.91 | −0.91 | −0.86 | −0.79 | −0.85 |
| Only | | | | | | | |
| Light - Lysol | Shirt | −0.96 | −0.96 | −0.91 | −0.91 | −0.79 | −0.8 |
| Only | (Clothes) | | | | | | |
| Light - Lysol + | Aluminum | 6.00 | 6.00 | 6.00 | 6.00 | 6.00 | 6.00 |
| ZnTPyP | | | | | | | |
| Light - Lysol + | Granite | 6.00 | 6.00 | 6.00 | 6.00 | 6.00 | 6.00 |
| ZnTPyP | Countertop | | | | | | |
| Light - Lysol + | PE | 6.00 | 6.00 | 6.00 | 6.00 | 6.00 | 6.00 |
| ZnTPyP | | | | | | | |
| Light - Lysol + | Aluminum | 6.00 | 6.00 | 6.00 | 6.00 | 6.00 | 6.00 |
| ZnTPyP | | | | | | | |
| Light - Lysol + | Granite | 6.00 | 6.00 | 6.00 | 6.00 | 6.00 | 6.00 |
| ZnTPyP | Countertop | | | | | | |
| Light - Lysol + | PE | 6.00 | 6.00 | 6.00 | 6.00 | 6.00 | 6.00 |
| ZnTPyP | | | | | | | |
| Light - Lysol + | Aluminum | 6.00 | 6.00 | 6.00 | 6.00 | 6.00 | 6.00 |
| ZnTPyP | | | | | | | |

Data = mean +/− SEM; N = 3. Bacteria were seeded at 10⁶ CFU on each sample. In Table 4, PE refers to Polyethylene, PU refers to polyurethane, PP refers to Polypropylene.

As shown in the Table 4 above, when a substrate surface was coated with only Lysol, bacteria increased in some instances after one week from the application of the Lysol which is indicated by a negative log reduction. However, when a Lysol formulation included porphyrins of the present disclosure, there was significant bacteria inhibition on the Lysol+porphyrin coated surfaces one week after exposure. The significant bacteria inhibition on Lysol+porphyrin coated surfaces one week after exposure occurred in both dark and light conditions.

### Examples

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### Example 1 Bacteria Inhibition

Through the below experiments, we have demonstrated that ZnTPyP, AgTPP, F-DCP or FeTCP reduce or completely eliminate bacteria colonization at concentrations as low as 0.0001 mg/ml when coated on numerous materials (such as aluminum, polypropylene, granite, tablecloths, polyethylene, shirt, polyurethane coated wood, and carpet) from 10 minutes to 3 weeks.

The synthesis of ZnTPyP nanoparticles and assays thereof will be illustrated. Specifically, 0.45 mL of a fresh stock ZnTPyP solution (0.01 M ZnTPyP dissolved in a 0.05 M HCl solution and stirring the mixture for 30 min) was prepared and quickly added into 9.1 mL of a continuously stirred aqueous solution of Cetyl trimethylammonium bromide (CTAB) (0.011M) and Sodium hydroxide (NaOH) (0.0027 M) at room temperature (about 25°) C. Zinc meso-tetra (4-pyridyl) porphyrin (ZnTPyP) is available from Frontier Scientific. Then, the mixture was stirred for 24 hrs. The green solution was centrifuged at 12000 rpm and washed twice with Millipore water to remove the surfactants. ZnTPyP nanoparticles were dispersed in water and then sterilized by UV light. Drops of 1 ml of the sterilized dispersion were added to surfaces having an area of 1 cm² (e.g., 1 ml drop per 1 cm² surface) at various concentrations (from 1 mg/ml to 0.0001 mg/ml) using standard pipetting techniques where 1 ml drops of the ZnTPyP containing solution described above were simply added to the surfaces and allowed to spread at room temperature.

Standard bacteria assays were then completed. Specifically, bacteria (Escherichia coli (ATCC 25922), multi-drug resistant (MDR) Escherichia coli (ATCC 8739), methicillin-resistant S. aureus (MRSA) (ATCC 4330), S. aureus (ATCC), Staphylococcus epidermidis (ATCC® 14990), Candida albicans (ATCC 10231), and Pseudomonas aeuroginosa (ATCC 9072)) at various concentrations (10⁶ to 1011 colony forming units) were separately pipetted onto each surface and cultured in standard conditions (37° C., humidified, 95%/5% air/CO₂ incubators in tryptic soy broth) for 5 minutes to 2 weeks either exposed to visible light (400 to 700 nm) or in the dark. The cultures were kept on agar plates at 4° C.

For this, bacteria were introduced into 6 mL of a sterile Luria-Bertani (LB) (bioPLUS, bioWORLD) medium in a 15 mL Falcon centrifuge tube and incubated at 37° C./200 rpm for 16 h. The optical density (OD) of the bacterial cultures was measured at 600 nm using a spectrophotometer (SpectraMax M3, Molecular Devices, Sunnyvale, CA). The bacterial suspension was then diluted to a concentration of 10⁶ or 1011 colony forming units per milliliter (CFU mL-1) and stored at 4° ° C. until seeded on the samples. For this, bacteria at the appropriate concentrations in TSB media were placed inside 6-well plates together with the different samples and they were placed inside an incubator for various time periods up to 2 weeks. Right after this time, for colony counting, samples were taken out and the bacterial media were removed. Samples were rinsed twice with phosphate-buffered saline (PBS) and each of them was transferred inside a 15 mL Falcon tube with 5 mL of PBS. The tubes were then placed inside an ultrasound water bath and sonication was applied for around 5 min. All bacteria were removed during this process. Afterwards, a suitable volume of each solution was extracted with micropipettes and diluted in sterile PBS to obtain serial dilutions of 100×. Then, 10 μL of each sample was placed onto a labeled agar plate and incubated for about 12 h at 37° C. The total number of colonies for each sample was counted post-incubation.

Nanoporphyrins of FeTCP, AgTPP, and F-DCP were similarly prepared. The data in Table 2 show that ZnTPyP, AgTPP, F-DCP or FeTCP nanoparticles at a very low concentration and coating significantly or complete inhibited bacteria after various exposure periods. Significant inhibition were observed under both light and dark conditions. These results were achieved with surfaces coated with 0.0001 mg/cm² (0.1 μg/cm²) and 0.0001 mg/cm² (1 μg/cm²) of the nanoparticles.

### Example 2: Virus Inhibition

Through the below experiments, we have demonstrated that ZnTPyP, AgTPP, F-DCP or FeTCP reduced or completely eliminated virus attachment at concentrations as low as 0.0001 mg/ml when coated on numerous materials (such as aluminum, polypropylene, granite, tablecloths, polyethylene, shirt, polyurethane coated wood, and a carpet) after 10 minutes to 3 weeks.

The following is an example of preparing the ZnTPyP nanoparticles. A mixture was prepared by quickly adding 0.45 mL of a fresh stock ZnTPyP solution (0.01 M ZnTPyP dissolved in a 0.05 M HCl solution and stirring the mixture for 30 min) into 9.1 mL of a continuously stirred aqueous solution of Cetyl trimethylammonium bromide (CTAB) (0.011M) and Sodium hydroxide (NaOH) (0.0027 M) at room temperature (25°) C. Then, the mixture was stirred for 24 hrs. The green solution was centrifuged at 12000 rpm and washed twice with Millipore water to remove the surfactants. ZnTPyP were sterilized by UV light and added to the surfaces (1 cm²) at various concentrations (from 1 mg/ml to 0.0001 mg/ml) using standard pipetting techniques where 1 ml drops of the ZnTPyP containing solution described above were simply added to the surfaces and allowed to spread at room temperature.

Standard virus attachment assays were then completed. Specifically, viruses (SARS-COV-2 (ATCC VR-1986 HK), Influenza A (ATCC 1895), Rhinovirus (ATCC VR-1185) and Norovirus (ATCC VR-1937)) were pipetted to the samples of interest at 10⁶ copies in a sterilized enclosed environment for 5 minutes to 4 hours. For the samples labeled 5 weeks, the coatings and control (uncoated) materials were allowed to sit on the shelf 5 weeks and then the viruses were added to the samples for 4 hours. At the end of that time period, the samples were soaked in a 1% wt. crystal violet stain (Sigma) for 1 hour. Then, the supernatant was removed and rinsed with PBS three times. At that time, 60% ethanol was added, and the crystal violet stain was eluted, collected, and run through a spectrophotometer. Color intensity was measured and compared to a standard curve to determine how much of the virus attached to the materials. Controls which consisted of no viruses were subtracted from values measured with the virus.

The data in Table 1 show that ZnTPyP, AgTPP, F-DCP or FeTCP nanoparticles at a very low concentration and coating significantly or completely inhibited virus attachment after 10 minutes to 3 weeks. Significant inhibition were observed under both light and dark conditions These results were achieved with surfaces coated with 0.0001 mg/cm² (0.1 μg/cm²) of ZnTPyP nanoparticles.

### Example 3: Zinc Nanoporphyrin (ZnTPyP) Bacteria Inhibition in Combination with a Quaternary Ammonium Disinfectant (Lysol)

Through the below experiments, we have demonstrated that the zinc meso-tetra (4-pyridyl) porphyrin (ZnTPyP) when added to a quaternary ammonium based disinfectant (Lysol) completely reduced or eliminated bacteria colonization at concentrations as low as 0.001 mg/ml when coated on numerous materials (such as aluminum, polypropylene, granite, tablecloths, polyethylene, shirt, polyurethane coated wood, and a carpet) from 5 minutes to 1 week.

Specifically, 0.45 mL of a fresh stock ZnTPyP solution (0.01 M ZnTPyP dissolved in a 0.05 M HCl solution and stirring the mixture for 30 min) was quickly added into 9.1 mL of a continuously stirred aqueous solution of cetyl trimethylammonium bromide (CTAB) (0.011M) and sodium hydroxide (NaOH) (0.0027 M) at room temperature (25°) C. Then, the mixture was stirred for 24 hrs. The green solution was centrifuged at 12000 rpm and washed twice with Millipore water to remove the surfactants. ZnTPyP were sterilized by UV light and added to a Lysol formulation at 0.001 mg/ml and then Lysol formulations with and without the ZnTPyP were applied to surfaces (1 cm²) using standard pipetting techniques. Drops of 1 ml drops of the formulations were simply added to the surfaces and allowed to spread at room temperature. The Lysol samples contained water, Ethanolamine, acid yellow 23, Lauramine Oxide, Phenoxyisopropanol, Alkyl (67% C12, 25% C14, 7% C16, 1% C8-C10-C18) dimethyl benzyl ammonium chloride, and Alkyl (50% C14, 40% C12, 10% C16) dimethyl benzyl ammonium chloride.

Standard bacteria assays were then completed. Specifically, bacteria (Escherichia coli (ATCC 25922), multi-drug resistant (MDR) Escherichia coli (ATCC 8739), methicillin-resistant S. aureus (MRSA) (ATCC 4330), S. aureus (ATCC), Staphylococcus epidermidis (ATCC® 14990), Candida albicans (ATCC 10231), and Pseudomonas aeuroginosa (ATCC 9072)) at 10⁶ colony forming units) were pipetted onto each surface and cultured in standard conditions (37° C., humidified, 95%/5% air/CO₂ incubators in tryptic soy broth) for 5 minutes to 1 week either exposed to visible light (400 to 700 nm) or in the dark. The cultures were kept on agar plates at 4° C.

For this, bacteria were introduced into 6 mL of a sterile Luria-Bertani (LB) (bioPLUS, bioWORLD) medium in a 15 mL Falcon centrifuge tube and incubated at 37° C./200 rpm for 16 h. The optical density (OD) of the bacterial cultures was measured at 600 nm using a spectrophotometer (SpectraMax M3, Molecular Devices, Sunnyvale, CA). The bacterial suspension was then diluted to a concentration of 10⁶ colony forming units per milliliter (CFU mL-1) and stored at 4° ° C. until seeded on the samples. For this, bacteria at the appropriate concentrations in TSB media were placed inside 6-well plates together with the different samples and they were placed inside an incubator for various time periods up to 4 hours. For the samples labeled 1 week, the Lysol and Lysol containing ZnTPyP formulations were coated a week prior to adding the bacteria for 4 hours. Right after this time, for colony counting, samples were taken out and the bacterial media were removed. Samples were rinsed twice with phosphate-buffered saline (PBS) and each of them was transferred inside a 15 mL Falcon tube with 5 mL of PBS. The tubes were then placed inside an ultrasound water bath and sonication was applied for around 5 min. All bacteria were removed during this process. Afterwards, a suitable volume of each solution was extracted with micropipettes and diluted in sterile PBS to obtain serial dilutions of 100×. Then, 10 μL of each sample was placed onto a labeled agar plate and incubated for about 12 h at 37° C. The total number of colonies for each sample was counted post-incubation.

No bacteria could be detected on samples with Lysol formulations with or without ZnTPyP nanoparticles either exposed to light or samples kept in the dark after 5 minutes of application of the formulations. That is, Lysol formulations effectively eliminated bacteria 5 minutes after application of the formulations. However, formulations that included ZnTPyP nanoparticles significantly reduced or even eliminated bacteria one week after the formulation was applied compared to formulations without ZnTPyP nanoparticles. The data for these results are shown in Table 4. These results were achieved with surfaces coated with 0.001 mg/cm² (1 g/cm²) of ZnTPyP nanoparticles. These data show that combining a fast acting disinfectant such as a quaternary ammonium compound (e.g., a benzalkonium chloride) together with porphyrin nanoparticles can effectively reduce and/or inhibit bacteria over an extended period of time, e.g., over a week.

### Example 4: Iron Chloride (FeCl) Nanoporphyrin, Silver Nanoporphyrin (AgTPP) and Fluorine Nanoporphyrin (F-DCP)

Through the below experiments, we have demonstrated that other nanoporphyrins, specifically iron (III) meso-tetra(4-carboxyphenyl)porphine chloride (FeTCP), silver (II) meso-tetraphenyl porphine (silver nanoporphyrins (AgTPP)) and 5,15-di(Pentafluorophenyl)-10,20-di(2-carboxyphenyl) porphine (fluorine nanoporphyrins (F-DCP)), reduced or completely eliminated bacteria colonization at concentrations as low as 0.0001 mg/ml when coated on numerous materials (such as aluminum, granite, polypropylene, and a carpet) after 5 minutes. Each of FeTCP, AgTPP, and FDCP are available from Frontier Scientific.

Specifically, 0.45 mL of a fresh stock either an Iron chloride (FeTCP), Silver (AgTPP) or Fluorine (F-DCP) solution (either 0.01 M AgTPP or F-DCP separately dissolved in a 0.05 M HCl solution and stirring the mixture for 30 min) was quickly added into 9.1 mL of a continuously stirred aqueous solution of Cetyl trimethylammonium bromide (CTAB)) (0.011M) and Sodium hydroxide (NaOH) (0.0027 M) at room temperature (25)° ° C. Then, the mixture was stirred for 24 hrs. The green solution was centrifuged at 12000 rpm and washed twice with Millipore water to remove the surfactants. The nanoporphyrins were sterilized by UV light and added at 0.0001 mg/ml (0.1 μg/ml) to the surfaces (1 cm²) using standard pipetting techniques where 1 ml drops of the nanoparticle containing solutions described above were simply added to the surfaces and allowed to spread at room temperature.

Standard bacteria assays were then completed. Specifically, bacteria (Escherichia coli (ATCC 25922), multi-drug resistant (MDR) Escherichia coli (ATCC 8739), methicillin-resistant S. aureus (MRSA) (ATCC 4330), and S. aureus (ATCC)) at 10⁶ colony forming units) were pipetted onto each surface and cultured in standard conditions (37° ° C., humidified, 95%/5% air/CO₂ incubators in tryptic soy broth) for 5 minutes either exposed to visible light (400 to 700 nm) or in the dark. The cultures were kept on agar plates at 4° C.

For this, bacteria were introduced into 6 mL of a sterile Luria-Bertani (LB) (bioPLUS, bioWORLD) medium in a 15 mL Falcon centrifuge tube and incubated at 37° C./200 rpm for 16 h. The optical density (OD) of the bacterial cultures was measured at 600 nm using a spectrophotometer (SpectraMax M3, Molecular Devices, Sunnyvale, CA). The bacterial suspension was then diluted to a concentration of 10⁶ colony forming units per milliliter (CFU mL-1) and stored at 4° C. until seeded on the samples. For this, bacteria at the appropriate concentrations in TSB media were placed inside 6-well plates together with the different samples and they were placed inside an incubator for various time periods up to 5 minutes. Right after this time, for colony counting, samples were taken out and the bacterial media were removed. Samples were rinsed twice with phosphate-buffered saline (PBS) and each of them was transferred inside a 15 mL Falcon tube with 5 mL of PBS. The tubes were then placed inside an ultrasound water bath and sonication was applied for around 5 min. All bacteria were removed during this process. Afterwards, a suitable volume of each solution was extracted with micropipettes and diluted in sterile PBS to obtain serial dilutions of 100×. Then, 10 μL of each sample was placed onto a labeled agar plate and incubated for about 12 h at 37° C. The total number of colonies for each sample was counted post-incubation.

Results showed similar or better bacteria inhibition when using either AgTPP or F-DCP compared to the aforementioned ZnPyP after 5 minutes (FIG. 3) and 1 week culture (FIG. 4) in the light and dark.

Only the preferred embodiment of the present invention and examples of its versatility are shown and described in the present disclosure. It is to be understood that the present invention is capable of use in various other combinations and environments and is capable of changes or modifications within the scope of the inventive concept as expressed herein. Thus, for example, those skilled in the art will recognize, or be able to ascertain, using no more than routine experimentation, numerous equivalents to the specific substances, procedures and arrangements described herein. Such equivalents are considered to be within the scope of this invention and are covered by the following claims.

1. A method of reducing or inhibiting growth of a virus, the method comprising applying a formulation including porphyrin nanoparticles to a surface to reduce or inhibit the growth of the virus on the surface.

2. The method of claim 1, wherein the virus is one of a SARS, SARS-COV-2, an Influenza, a Rhinovirus and/or a Norovirus.

3. The method of claim 1, wherein applying the formulation including porphyrin nanoparticles to the surface imparts a residue of porphyrin nanoparticles on the surface having an antimicrobial activity (with and without visible light) to reduce or inhibit the growth of the virus on the surface for a period of at least 24 hours after application of the formulation.

4. A method of reducing or inhibiting growth of a broad spectrum of infectious microbes, the method comprises applying a formulation including porphyrin nanoparticles to a surface to reduce or inhibit the growth of the at least two infectious microbes, wherein at least one of the infectious microbes is a multi-drug resistant bacteria.

5. The method of claim 4, wherein the at least one multi-drug resistant bacteria is a multi-drug resistant (MDR) Escherichia coli or methicillin-resistant S. aureus.

6. The method of claim 4, wherein applying the formulation including porphyrin nanoparticles to the surface imparts a residue of porphyrin nanoparticles on the surface having an antimicrobial activity (with and without visible light) to reduce or inhibit the growth of the virus on the surface for a period of at least 24 hours after application of the formulation.

7. (canceled)

8. The method of claim 1, wherein applying the formulation reduces or inhibits the growth of the virus on the surface for a period of at least one week after application of the formulation.

9. The method of claim 1, wherein the formulation further includes a fast acting disinfectant which is different from the porphyrin nanoparticles.

10. The method of claim 1, wherein the porphyrin nanoparticles are included in the formulation at a concentration from 10⁶ μg/ml to about 1 mg/ml.

11. The method of claim 1, wherein the porphyrin nanoparticles comprise an iron-based porphyrin nanoparticles.

12. A formulation comprising (i) a fast acting disinfectant and (ii) porphyrin nanoparticles at a concentration from about 10⁻⁶ μg/ml up to about 1 mg/ml, wherein application of the formulation to a surface imparts a residue of porphyrin nanoparticles on the surface having antimicrobial activity (with and without visible light) on the surface for at least 24 hour after application of the formulation.

13. The formulation of claim 12, wherein the microbes include one or more viruses.

14. The formulation of claim 12, wherein the disinfectant comprises one or more quaternary ammonium compounds.

15. The formulation of claim 12, wherein the porphyrin nanoparticles comprise iron-based porphyrin nanoparticles.
